(19) United States
(12) Patent Application Publication
(10) Pub. No.: US 2021/0205791 A1
(43) Pub. Date: Jul. 8, 2021

(54) METHOD OF MAKING NANOMETER THIN SHEETS OF METALS IN AIR

(71) Applicant: INDIAN INSTITUTE OF TECHNOLOGY MADRAS (IIT MADRAS), Chennai (IN)

(72) Inventors: Pradeep THALAPPIL, Chennai (IN); Depanjan SARKAR, Chennai (IN); Anirban SOM, Chennai (IN); Manju C.K, Chennai (IN)

(21) Appl. No.: 16/755,395

(22) PCT Filed: Oct. 12, 2018
(86) PCT No.: PCT/IN2018/050654

§ 371 Date: Apr. 10, 2020

(30) Foreign Application Priority Data
Oct. 12, 2017 (IN) 201741036233

(51) Int. Cl. B01J 23/52; B01J 23/44; B01J 23/50; B01J 35/00; B01J 37/34; B01J 37/16

(52) U.S. Cl. CPC B01J 23/52; B01J 23/44; B01J 37/16; B01J 35/0013; B01J 37/347; B01J 23/50

(57) ABSTRACT

The present invention relates to an ambient ion based method of making free-standing 2D metal sheets made of bare NPs, at the air-liquid interface. An electro-hydrodynamic flow field was generated by electrospray deposition on the liquid surface, which in turn assisted the assembly of the NPs. The NP-NSs were made under ambient conditions at room temperature from metal salt precursors. The sheets can be made of different elements such as Pd, Au, and Ag. Synthesized 2D NP-NSs were used as efficient and reusable heterogeneous catalysts for C—C bond formation reactions. These thin metal sheets may also be used as catalysts, sensors, gas adsorbing media, electrodes for electrochemical reactions, etc.

### FIELD OF THE INVENTION

The present invention relates to a method of making nanometer thin sheets of metals in air. More specifically relates to an ambient ion based method of making free-standing 2D metal sheets made of bare nanoparticles, at the air-liquid interface. The resulting free-standing nanoparticle-nanosheet was used for applications such as heterogeneous catalysis for C—C bond formation it may be used as a catalyst, sensor, gas adsorbing medium, electrode for electrochemical reactions, etc.

### BACKGROUND OF THE INVENTION

Molecular interactions at liquid-air interfaces have been investigated from the times of Agnes Pockels [Pockel A et al., Phys. Z. 1914, 15, 39-46; Reich K et al., ActaCrystallogr., Sect. A: Found. Crystallogr. 2008, 64 (3), 432]. Assembled structures at the air-liquid interfaces subsequently transferred to solid surfaces have contributed to the understanding of two-dimensional (2D) films of diverse materials, including nanoparticles (NPs) [Natansohn A et al., Chem. Rev. (Washington, D.C., U. S.) 2002, 102 (11), 4139-4175; Li X et al., Nat. Nanotechnol. 2008, 3 (9), 538-542; Cote L. J et al., J. Am. Chem. Soc. 2009, 131 (3), 1043-1049; Kim J et al., J. Am. Chem. Soc. 2010, 132 (23), 8180-8186; Whang D et al., Nano Lett. 2003, 3 (9), 1255-1259; Fendler, J. H et al., Chem. Mater. 1996, 8 (8), 1616-1624; Kim, F et al., J. Am. Chem. Soc. 2001, 123 (18), 4360-4361; Lu, Y et al., Nano Lett. 2005, 5 (1), 5-9; Zasadzinski J. A, et al., Science (Washington, D.C., 1883-) 1994, 263 (5154), 1726-33; Hammond P. T, et al., Adv. Mater. (Weinheim, Ger.) 2004, 16 (15), 1271-1293]. While stable molecules and particles arrange at the interface due to surfactancy, it is possible to create nanostructures at the interface directly, starting from atomic precursors. A new methodology introduced recently to synthesize metal NPs on solid surfaces by ambient electrolytic spray [Li A et al., Angew. Chem., Int. Ed. 2014, 53 (12), 3147-3150] as well as electrospray [Sarkar D et al., Adv. Mater. (Weinheim, Ger.) 2016, 28 (11), 2223-2228] can be adapted to liquid surfaces leading to synthesis and assembly simultaneously. The presence of an electrical double layer at the air-liquid interface and its mobility in response to moderate electric fields can drive motion at both the surface and the bulk of the liquid which, in turn, can guide suspended NPs into ordered assemblies. Generally, thin sheets of metals are made in vacuum. Thin film technology is highly advanced to develop materials of different kinds. Thermal evaporation [Shen Z et al., Science (Washington, D.C.) 1997, 276 (5321), 2009-2011], electron beam evaporation [Sheu J. K et al., Appl. Phys. Lett. 1999, 74 (16), 2340-2342; Menard E et al., Langmuir 2004, 20 (16), 6871-6878], magnetron sputtering [Zoppi G et al., Prog. Photovoltaics 2009, 17 (5), 315-319] and several other methods are used to accomplish this. All these methods require either of the conditions like elevated temperature, high vacuum, sophisticated instrumentation, etc.

The present invention provides an ambient method of making thin films of metals by depositing nanometer scale droplets on liquid surfaces, directed in an electric field. With this objective, a series of experiments are performed by which NPs of Pd were synthesized on the surface of a water reservoir which then self-assembled to form nanoparticle-nanosheets (NP-NSs).

### SUMMARY OF THE INVENTION

The present invention relates to a method of making thin films of metals in air by depositing nanometer scale droplets on liquid surfaces, directed in an electric field. More particularly it relates to the synthesis of self-assembled thin sheets of palladium nanoparticles on the surface of a water reservoir.

In one embodiment, a series of experiments are performed by which NPs of Pd are synthesized on the surface of a water reservoir using electrospray which then self-assembled to form nanoparticle-nanosheets (NP-NSs). Electrospraying of palladium chloride was conducted in acetonitrile over a water reservoir, the electrospraying was produced at a voltage of 1000-2000 V, at a distance of 10-15 mm from the liquid surface. Visualization of the surface and bulk motion of the liquid, using coloured dyes, suggested that the fluid flow was the principal mechanism underlying this spontaneous self-assembly. The resulting free-standing NP-NS was used for applications such as heterogeneous catalysis for C—C bond formation. Furthermore this thin metal film was used as a catalyst, sensor, gas adsorbing medium, electrodes for electrochemical reactions, etc. The simplicity and versatility of this methodology allows for diverse precursors and varying liquids, opens up the possibility of creating a rich variety of materials and studying novel physico-chemical phenomena. The solvent used for the precursor solutions can be methanol, ethanol, water, acetonitrile and mixture thereof with different proportions. The liquids used in the reservoir can be water, ethylene glycol, ionic liquids or liquid metals or semiconductors which are solids at room temperature.

In another embodiment, the present invention provides an ambient ion based method of making free-standing 2D metal sheets made of bare NPs, at the air-liquid interface. An electro-hydrodynamic flow field was generated by electrospray deposition on the liquid surface, which in turn assisted the assembly of the NPs. The NP-NSs were made under ambient conditions at room temperature from metal salt precursors. The sheets can be made of different elements such as gold, silver, platinum, palladium, nickel, copper and of different alloys like silver-palladium, gold-palladium, etc. The deposition of films by electrosprays and solvent evaporation of the droplets are achieved along with other excitations such as light, temperature, electric and magnetic fields, etc. Synthesized 2D NP-NSs were used as efficient and reusable heterogeneous catalysts for C—C bond formation reactions.

### BRIEF DESCRIPTION OF THE DRAWINGS

FIG. 1A) Schematic of the electrospray deposition of PdCl2 on water surface, B) mass spectrum of PdCl2 solution in acetonitrile, collected using nESI source and C) optical photograph of electrospray deposition at air-water interface. The electrospray was visualized using a green laser pointer showing scattering from the droplets. Liquid was grounded by a metal clip. D), E) TEM images of the formed Pd NP-NS collected from the liquid surface after washing with water, at different magnifications. Inset of E) shows HRTEM image of Pd sheet showing that it is made of nearly uniform crystalline Pd NPs and a size distribution histogram for the Pd NPs.

FIG. 2A) and B) TEM image of Pd NP-NS synthesized by slow (30 nA) and fast (80 nA) electrospray deposition, respectively.

FIG. 3 A) EDS mapping and spectrum of the Pad NP-NSs, showing the presence of only Pd, B) X-ray photoelectron spectrum showing that Pd is in its zero valence state with slight contribution from a surface oxide layer, C) UV-Vis spectra of Pd NP-NS, water after Pd deposition and PdCl₂ in ACN. For the Pd sheet, as there is scattering, the Y axis may be taken as extinction. D) and E) AFM images of the Pd NP-NSs and F) height profile taken across the red line in E. 3D views are shown to illustrate the roughness. The image was taken deliberately by including a crack in the film to measure the film thickness. The film was taken on a silicon wafer.

FIG. 4 Plot of conductivity of the water, on which deposition happened vs deposition time, showing linear enhancement in conductivity.

FIG. 5 A) TEM image of as synthesized Pd NP-NSs over 80 μm2 area, B) Pd NP-NS after washing with DI water.

FIGS. 6 A) and B) TEM images of NP-NSs made of silver and gold, respectively.

FIG. 7 Mass spectrum collected after Pd NP-NS catalyzed coupling reaction between 4-tolylboronic acid and 4-bromophenol, inset shows the structures of the reactants and the product.

FIG. 8 A) Mass spectrum collected from the reaction mixture of p-tolylboronic acid and p-bromophenol without catalyst, B) with 1.3 mg commercial catalyst, and C) with Pd NP-NS catalyst.

FIG. 9 Mass spectrum collected from the reaction mixtures (serial no. 2-5) mentioned in Table 1.

FIG. 10 Mass spectrum collected from the reaction mixtures (serial no. 6-10) mentioned in Table 1.

FIG. 11 UV-Vis spectrum collected for the reactants and product. The dashes and dotted traces are UV-Vis spectra of 4-iodophenol and 4-tolylphenylboronic acid, respectively.

FIG. 12A) UV-Vis spectra of Pd NP-NS before and after the catalysis reaction, showing that catalysis does not change the nature of the sheet, B) intensity (absolute intensity of the product in mass spectrum) vs number of cycles for the same catalyst, showing that after 6 cycles also 81% of the catalytic activity was restored, C and D) TEM images of the Pd NP-NS after reaction showing that the nature of the sheet remains almost unchanged, inset in D shows the EDS spectrum taken from the same.

Referring to the drawings, the embodiments of the present invention are further described. The figures are not necessarily drawn to scale, and in some instances the drawings have been exaggerated or simplified for illustrative purposes only. One of ordinary skill in the art may appreciate the many possible applications and variations of the present invention based on the following examples of possible embodiments of the present invention.

### DETAILED DESCRIPTION OF THE INVENTION

The present invention relates to a method of making thin films of metals in air by depositing nanometer scale droplets on liquid surfaces, directed in an electric field. More particularly relates to synthesize of self-assembled thin sheet of palladium nanoparticle on the surface of a water reservoir.

The present invention illustrates a series of experiments by which nanoparticles of Pd is synthesized on the surface of a water reservoir using electrospray which then self-assembled to form nanoparticle-nanosheets (NP-NSs). Electrospraying of palladium chloride in acetonitrile over the water reservoir was conducted at a voltage 1000-2000 V, at a distance 10-15 mm from the liquid surface. Visualization of the surface and bulk motion of the liquid, using colored dyes, suggested that the fluid flow is the principal mechanism underlying this spontaneous self-assembly. The resulting free-standing NP-NS was used for applications such as heterogeneous catalysis for C—C bond formation. Furthermore this thin metal film is used as a catalyst, sensor, gas adsorbing medium, electrodes for electrochemical reactions, etc. The simplicity and versatility of this methodology allows for diverse precursors and varying liquids, opens up the possibility of creating a rich variety of materials for studying novel physico-chemical phenomena. The solvent for the precursor solution can be methanol, ethanol, water, acetonitrile and a mixture thereof with different proportions. The liquids used in the reservoir can be water, ethylene glycol, ionic liquids or liquid metals or semiconductors which are solids at room temperature.

The present invention provides an ambient ion based method of making free-standing 2D metal sheets made of bare NPs, at the air-liquid interface. An electro-hydrodynamic flow field was generated by electrospray deposition on the liquid surface, which in turn assisted the assembly of the NPs. The NP-NSs were made under ambient conditions at room temperature from metal salt precursors. The sheets can be made of different elements such as gold, silver, platinum, palladium, nickel, copper and different alloys like silver-palladium, gold-palladium, etc. The deposition of films by electrospray and solvent evaporation of the droplets can be achieved along with other excitations such as light, temperature, electric and magnetic field, etc. Synthesized 2D NP-NSs were used as efficient and reusable heterogeneous catalysts for C—C bond formation reactions.

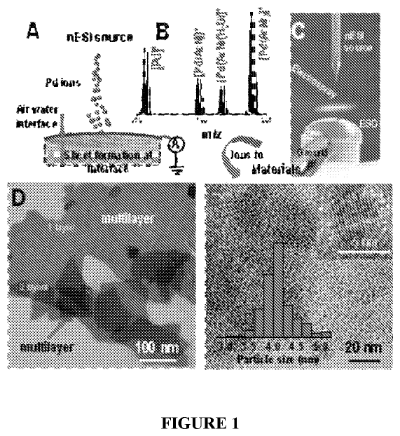

Experiments were conducted as shown in FIG. 1A, wherein a nanoelectrospray (nESI) source gently deposits ions (60-70 nA) on a grounded liquid surface at a voltage range between 1000-2000 V from 10-15 mm distance. At first, the collected mass spectrum (FIG. 1B) from an electrosprayed solution of PdCl₂ in acetonitrile (ACN) is shown. Peaks corresponding to Pd⁺ and solvated Pd⁺ ions such as [Pd(AcN)]⁺, [Pd(AcN)(H₂O)]⁺, [Pd(AcN)₂]⁺ confirm that the droplets generated in electrospray contain Pd in its +1 state. Electrospray, being reducing in nature, converts Pd(II) to Pd(I) within the charged droplets. These Pd⁺ ions were deposited on a grounded water surface. A copper strip was attached to the wall of the container and it was grounded through a picoammeter. In the course of deposition, Pd⁺ ions got reduced to Pd(0) by taking electrons from the grounded electrode. The deposited neutral Pd atoms aggregated to form Pd NPs. The monodispersity of the NPs suggests that the uniformity of the droplets has a significant role in controlling the particle size. The NPs were assumed to be born as soon as the charged droplets, containing similar number of ions, impacted the water surface. The electron transfer from the grounded electrode to the metal ions was assisted by the hydroxyl ions (OH⁻). FIG. 1C shows an optical photograph of the experimental set up. FIG. 1D shows a TEM image of clean Pd NP-NSs. Detailed TEM imaging was performed to prove that the sheet was made of Pd NPs. FIG. 1E shows a TEM image of the as-synthesized Pd NP-NSs showing the presence of monodisperse Pd NPs in the sheet. The average size of Pd NPs was around 4 nm. The inset in FIG. 1E shows a high-resolution transmission electron microscopic (HRTEM) image of the NPs present in the sheet. The lattice distance of these NPs match with the (111) plane of Pd, proving the metallic nature of the Pd NPs.

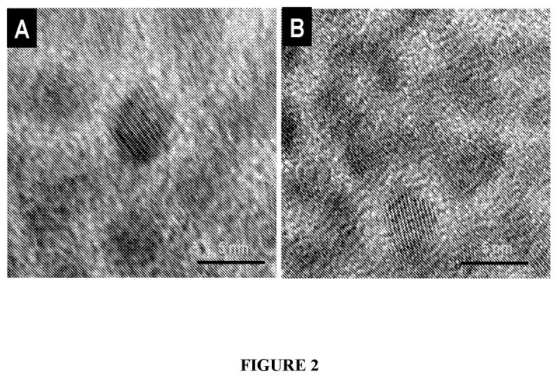

High mobility of the Off ions resulted in fast transfer of electrons, leading to the formation of uniform Pd NPs at the water surface. OH⁻ ions are also responsible for the formation of a mobile electrical double layer which drives an electrohydrodynamic flow at both the surface and the bulk of the water. The Pd NPs move in the flow field and arrange themselves to form a thin nanosheet at the air-water interface. Deposition at a very low deposition current i.e. a slow deposition rate leads to the formation of a better ordered assembly, whereas a fast deposition leads to a glassy assembly of the NPs. FIGS. 2 A and B show TEM images of Pd NP-NSs synthesized by slow (30 nA) and fast (80 nA) electrospray deposition conditions, respectively. Such currents can be carried by the residual Off ions present (6.023×10¹⁶ ions/L) in the neutral water. After 1 h of deposition at a deposition current 40 nA, an orange colored film was seen floating on water. This film was collected on different substrates, by scooping it from water and was characterized using various techniques.

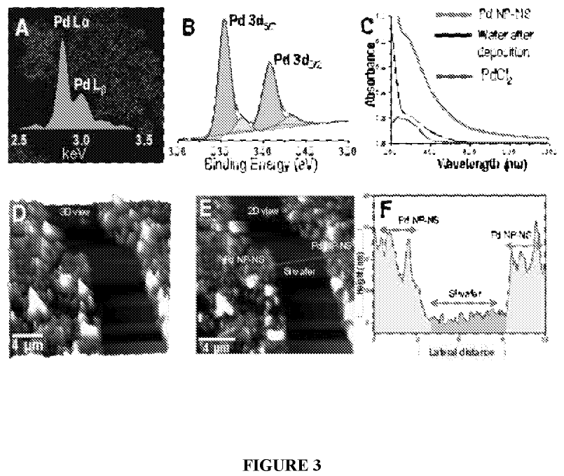

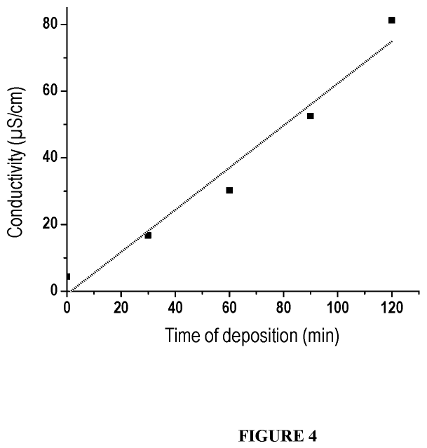

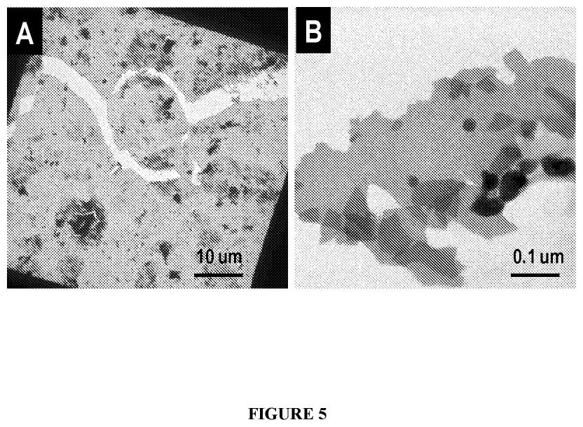

Energy dispersive spectroscopic (EDS) analysis of the same shows the presence of only Pd in the sheet (FIG. 3A). Hence we speculate that the chloride ions (counterpart of the precursor) go into the water reservoir. This was proved by measuring the conductivity of the water with respect to deposition time (FIG. 4), the plot shows a linear enhancement. FIG. 3B shows the X-ray photoelectron spectrum (XPS) of Pd NP-NSs with peaks due to Pd 3d_{5/2} (335.4 eV) and Pd 3d_{3/2} (340.7 eV), which support the presence of Pd(0). The XPS spectrum does not show the presence of any other species like chlorine proving that these particles are unprotected. For the XPS study, more material was collected on the sample plate by collecting the NP-NS multiple times. EDS spectrum collected from the Pd NP-NSs shows that the NSs are made of 100% Pd. There are no organic substances present as protecting species. Hence, we believe that the NPs are bare metal particles. We see the presence of small amounts of oxygen in EDS and a weak feature due to Pd²⁺ in XPS. This surface oxide may be the bridge for the NPs to form NSs, along with van der Waals forces. FIG. 3C shows the UV-Vis spectra of the Pd NP-NSs (red trace, solid state UV-Vis spectrum of Pd NP-NS, coated on a quartz coverslip), the water on which the deposition happened (black trace) and the precursor PdCl₂ solution (blue trace). The difference between the blue and red trace clearly proves that the salt solution has transformed to metal NPs upon ESD, at room temperature. The hump near 300 nm is a characteristic feature of Pd NPs due to the plasmonic excitation, which occurs around 340 nm for naked Pd NPs in a pure dispersion in toluene (0.6 mg/3 mL)[Chen S et al., Chem. Mater. 2000, 12 (2), 540-547]. The UV-Vis spectrum of the water after deposition (black trace) also shows the same feature corresponding to Pd NPs. This implies that some portion of Pd NPs goes into the water due to the generated flow field in water, whereas most of them stay at the interface and arrange to form Pd NP-NSs. A quantitative theory of this phenomenon is provided below. Atomic force microscopy (AFM) was performed on the Pd NP-NSs to understand the thickness. FIGS. 3D and E show the AFM images of the Pd NP-NSs, in 3D and 2D views, respectively. FIG. 3F shows a height profile of a Pd NP-NS showing an average thickness of 26 nm and a roughness of ±12 nm. This metallic sheet can be made over a large area; FIG. 5A shows a TEM image of such a NP-NS over an area of 80 μm². The image shows the presence of excess Pd on the sheet, due to longer deposition time, which can be removed by washing with DI water. FIG. 5B shows the TEM image of the same sheet after washing. In this case, it was observed that very clean sheets of Pd are made of Pd NPs. The sheet was broken into small parts after washing, retaining their morphology, proving the stability of these NP-NSs. Free-standing metal NP-NSs were made using other noble metals also.

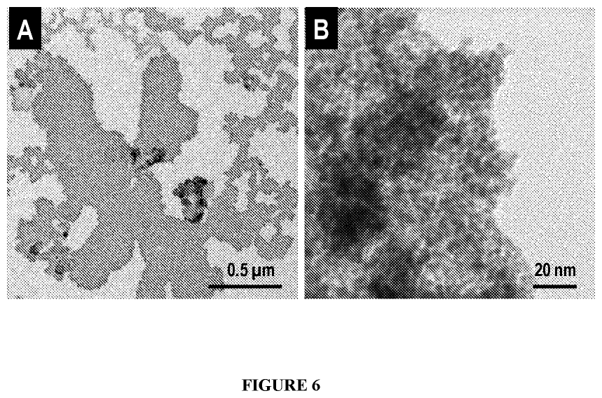

FIGS. 6A and B show the TEM images of NP-NSs made of silver and gold, respectively. In the case of gold, an acetonitrile solution of Au(III) acetate was electrosprayed on water. Just like the case of Pd, formation of a thin film was seen at the air-water interface. The thin film was collected on a TEM grid for characterization. In the case of Ag, the liquid was changed from water to ethylene glycol (EG), to ensure reduced dispersion of Ag NPs into the liquid, as an aqueous solution of AgOAc was used as the precursor for Ag. In contrast, when water was used as a deposition substrate, Ag NPs did not form the nanosheet. Hence, the solubility of the precursor salt and surface tension of the liquid substrate play crucial roles in the NP-NS formation.

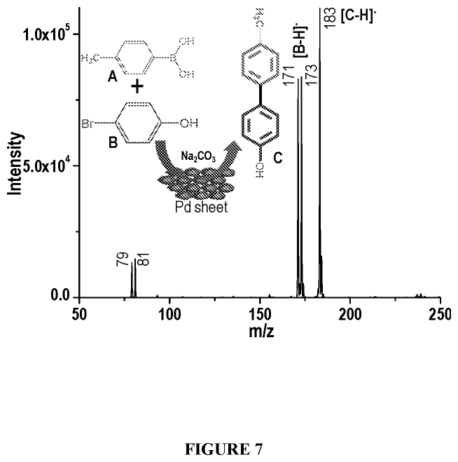

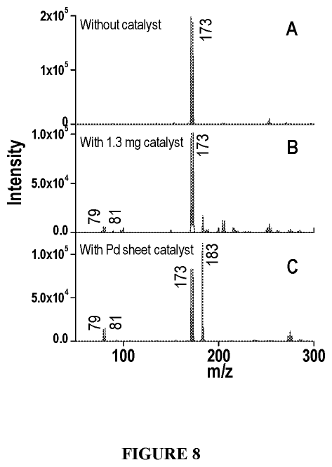

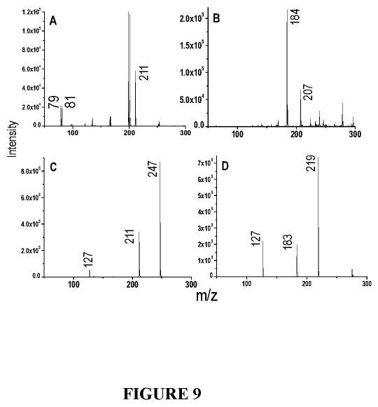

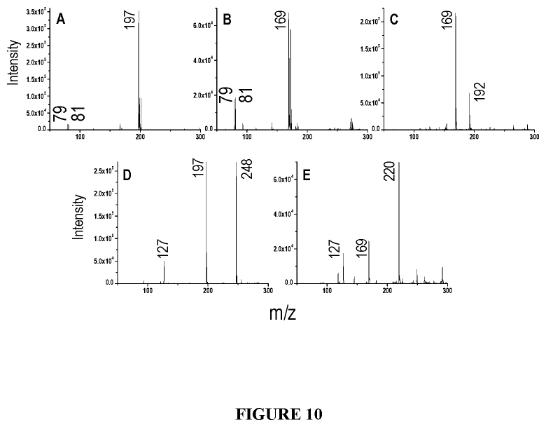

Pd in its zero oxidation state is very well known for catalyzing C—C bond formation reactions. Hence, the catalytic activity of the synthesized Pd NP-NS was tested for the Suzuki-Miyaura coupling reaction. For this, the as synthesized Pd NP-NSs were taken on quartz cover slips and dipped into a reaction mixture of a boronic acid and an organohalide. An aqueous solution of Na₂CO₃ was added to the reaction mixture to make it basic in nature. After the addition of the catalyst, the reaction mixture was stirred in a round bottom flask at room temperature and the product was analyzed using mass spectrometry. FIG. 7 shows a mass spectrum of the reaction mixture containing 4-bromophenol (reactant I) and 4-tolylboronic acid (reactant II) in presence of Pd NP-NS catalyst after 30 min of stirring. Intense peak at m/z 183 in the mass spectrum corresponds to the reaction product, i.e. 4′-methyl[1,1′-biphenyl]-4-ol (III). The inset shows the molecular structures of the reactants and the product. The mass spectrum also shows two peaks for II at m/z 171 and 173, due to the equal abundance of the isotopes of Br. Sodium bromide (NaBr) is a bi-product in this reaction leading to the presence of peaks corresponding to bromide ion (Br−) in the mass spectrum (peaks at m/z 79 Br− and 81 Br−). Control experiments were performed to prove that the Pd NP-NS catalyst was essential for the coupling reaction. The mass spectrum (FIG. 8A) collected from a mixture of the same reactants, without a catalyst, after 30 min did not show any peak other than the reactants. A comparative study was also carried out to estimate the efficiency of our catalyst. In this experiment, commercially available tetrakis(triphenylphosphine)palladium(0) was used as the catalyst for one set of reaction (reaction mixture X). In the other set (reaction mixture Y), Pd NP-NS was used as the catalyst. All the other parameters such as the concentration of the reactants, solvent, temperature and reaction time were kept constant in both the cases. FIGS. 8B and C show the mass spectrum collected from the reaction mixtures X and Y, respectively after 30 min of stirring. The efficiency (calculated considering only the metal percentage in both the cases) of Pd NP-NS catalyst was approximately 23 times higher than the commercial catalyst. The 2D nature and surface roughness exposing more catalytically active sites may be attributed as the main reason for higher efficiency. It was tested for C—C coupling reactions for various boronic acids and organic halides (Table 1, FIGS. 9, 10).

Table 1: Shows the chemical structures of reactants, products and m/z values of the products for all the entries.

| S .No | Reactant A | Reactant B | Product | m/z |
|---|---|---|---|---|
| 1 | 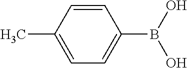 | 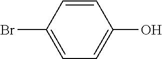 | 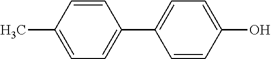 | 183 |
| 2 | 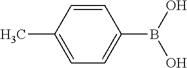 | 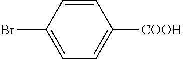 | 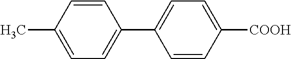 [SMILES: Cc1ccc(-c2ccc(C(=O)O)cc2)cc1] | 211 |
| 3 | 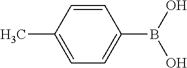 | 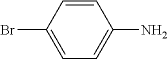 | 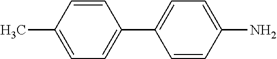 | 184 |
| 4 | 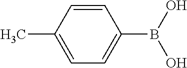 | 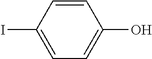 | 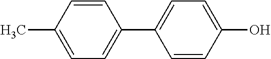 | 183 |
| 5 | 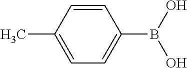 | 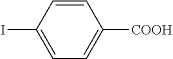 | 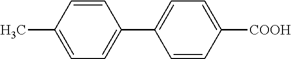 [SMILES: Cc1ccc(-c2ccc(C(=O)O)cc2)cc1] | 211 |
| 6 | 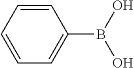 | 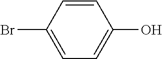 | 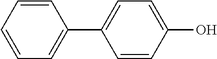 [SMILES: Oc1ccc(-c2ccccc2)cc1] | 169 |
| 7 | 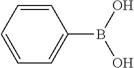 | 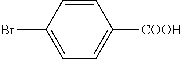 | 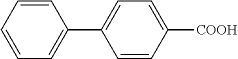 [SMILES: O=C(O)c1ccc(-c2ccccc2)cc1] | 197 |
| 8 | 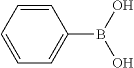 | 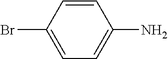 | 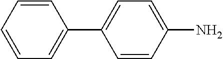 [SMILES: Nc1ccc(-c2ccccc2)cc1] | 169 |
| 9 | 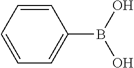 | 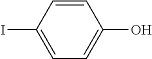 | 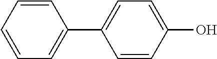 [SMILES: Oc1ccc(-c2ccccc2)cc1] | 169 |
| 10 | 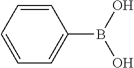 | 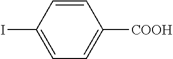 | 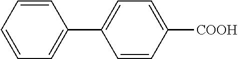 [SMILES: O=C(O)c1ccc(-c2ccccc2)cc1] | 197 |

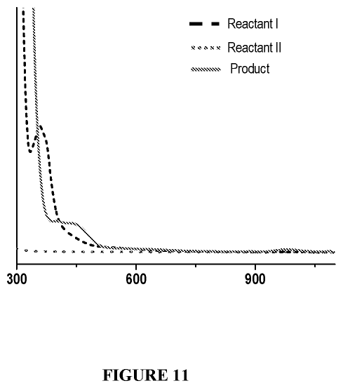

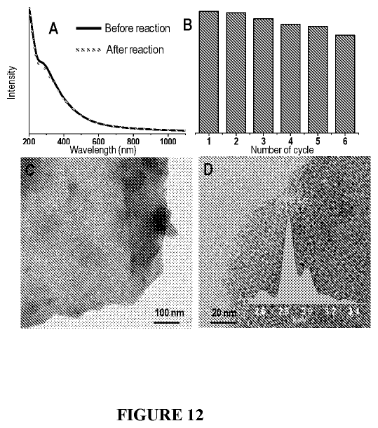

UV-Vis spectroscopy also supports the C—C bond formation showing a broad hump around 450 nm (the solid trace in FIG. 11), a characteristic feature of biphenyl compounds. All the data presented here prove that the Pd NP-NSs are more efficient catalysts for Suzuki-Miyaura coupling reaction. Stability of the catalyst was also checked using UV-Vis spectroscopy and TEM imaging. FIG. 12A shows the solid state UV-Vis spectrum of Pd NP-NS before and after catalysis. The spectrum was identical in both the cases. A small decrease in the intensity can be due to the loss of a small portion of Pd NP-NS while washing. Reusability of the catalyst was also checked. FIG. 13B shows a plot of the absolute intensity of the product formed when the same catalyst was used again for a particular coupling reaction. Efficiency of the catalyst can be calculated taking the absolute intensity of the peak, in the mass spectrum, corresponding to the product. Figure S8B shows that after 6 cycles of catalysis reaction, about 81% of the catalytic activity was retained. FIGS. 12C and D illustrate TEM images of the Pd NP-NSs after the reaction, showing that NPs are intact and are similar in size. EDS spectrum of the Pd NP-NS after the reaction exhibits only Pd. These data prove that the Pd NP-NS act as a catalyst for the coupling reactions and the same catalyst can be reused for many reactions.

Thus the present invention provides an ambient ion based method of making free-standing 2D metal sheets made of bare NPs, at the air-liquid interface. An electro-hydrodynamic flow field was generated by electrospray deposition on the liquid surface was responsible for the assembly of the NPs. This is the first report of generating such a flow field in fluids using electrospray deposition. The NP-NSs were made under ambient conditions at room temperature from metal salt precursors. The sheets can be made of different elements such as Pd, Au, and Ag. Synthesized 2D NP-NSs were used as efficient and reusable heterogeneous catalysts for C—C bond formation reactions.

It may be appreciated by those skilled in the art that the drawings, examples and detailed description herein are to be regarded in an illustrative rather than a restrictive manner.

We claim:

1. A method of making nanometer thin, <100 nm free standing 2D metal sheets made of bare nanoparticles at air-liquid interface, wherein the method comprises;
Electrospraying of at least one metal salt precursor solution in acetonitrile over a water reservoir, wherein the electrospray is produced at a voltage 1000-2000 V, at a distance of 10-15 mm from the liquid surface and
nanoparticles of metal are synthesized on the surface of a water reservoir which then self-assembled to form nanoparticle-nanosheets.

2. The method of making nanometer thin metal films as claimed in claim 1, wherein the metal is palladium.

3. The method of making nanometer thin metal films as claimed in claim 1, wherein the metal salt is palladium chloride.

4. The method of making nanometer thin metal films as claimed in claim 1, wherein the metals are selected from gold, silver, platinum, palladium, nickel and copper.

5. The method of making nanometer thin metal films as claimed in claim 1, wherein the metals are selected from various salts of gold, silver, platinum, palladium, nickel and copper.

6. The method of making nanometer thin metal films as claimed in claim 1, wherein the thin film is made of different alloys including silver-palladium and gold-palladium.

7. The method of making nanometer thin metal films as claimed in claim 1, wherein the solvent of the precursor solution includes but not limited to methanol, ethanol, water, acetonitrile and combination thereof with different proportions.

8. The method of making nanometer thin metal films as claimed in claim 1, wherein the liquid reservoir contain liquids including but not limited to water and ethylene glycol.

9. The method of making nanometer thin metal films as claimed in claim 1, wherein the liquid reservoir contains an ionic liquid.

10. The method of making nanometer thin metal films as claimed in claim 1, wherein the liquid reservoir contains liquid metals or semiconductors which are solids at room temperature.

11. The method of making nanometer thin metal films as claimed in claim 1, wherein the electrospray deposition of films occurs along with other stimuli including light, temperature and magnetic field.

12. The method of making nanometer thin metal films as claimed in claim 1, wherein the modification of the electrospray occurs during solvent evaporation of the droplets using temperature, light, electric and magnetic fields.

13. The method of making nanometer thin metal films as claimed in claim 1, wherein the thin metal films are used as a catalyst, sensor, gas adsorbing medium and electrodes for electrochemical reactions.

14. The method of making nanometer thin metal films as claimed in claim 1, wherein the atmosphere over the liquid surface is composed of specific gases such as nitrogen, oxygen, hydrocarbons, etc.

15. The method of making nanometer thin films as claimed in claim 1, wherein the prepared film is processed subsequently by washing, heating, etc.
